(19) United States
(12) Patent Application Publication
(10) Pub. No.: US 2024/0299325 A1
(43) Pub. Date: Sep. 12, 2024

(54) SUCCINIC ACID AND DERIVATIVES FOR THE TREATMENT OF HAEMOTOLOGICAL DISORDERS

(71) Applicant: ENZENE BIOSCIENCES LIMITED, Pune (IN)

(72) Inventors: Himanshu GADGIL, Pune (IN); Abir BANERJEE, Pune (IN); Arindam CHAKRABORTY, Pune (IN); Mazin ADENWALA, Pune (IN)

(73) Assignee: ENZENE BIOSCIENCES LIMITED, Pune (IN)

(21) Appl. No.: 18/667,471

(22) Filed: May 17, 2024

Related U.S. Application Data
(62) Division of application No. 17/254,391, filed on Dec. 21, 2020, now abandoned.

(30) Foreign Application Priority Data
Jun. 22, 2018 (IN) 201821023469

(51) Int. Cl. A61K 31/194; A61K 31/165; A61K 31/17; A61K 31/198; A61K 31/337; A61K 31/475; A61K 31/513; A61K 31/519; A61K 31/52; A61K 31/7048; A61K 31/7056; A61K 31/706; A61K 31/7068; A61K 31/7076; A61K 38/12; A61P 7/00

(52) U.S. Cl. CPC A61K 31/194; A61K 31/165; A61K 31/17; A61K 31/198; A61K 31/337; A61K 31/475; A61K 31/513; A61K 31/519; A61K 31/52; A61K 31/7048; A61K 31/7056; A61K 31/706; A61K 31/7068; A61K 31/7076; A61K 38/12; A61P 7/00

(57) ABSTRACT

The present invention relates to succinic acid and its derivative(s) of Formula (I), their pharmaceutically acceptable salts, polymorphs, solvates, hydrates, co-crystals, isomers and solvates thereof as an active pharmaceutical ingredient for the treatment of haematological disorder(s). The present invention further relates to compounds of Formula (I), for the treatment of disorders selected from dengue, idiopathic thrombocytopeniarpura (ITP), anaemia and chemotherapy induced myelosuppression. The present invention also relates to use of succinic acid for the treatment of haematological disorder(s), wherein succinic acid is extracted from Papaya leaves. The present invention also provides pharmaceutical compositions comprising succinic acid and its derivative(s) of Formula I and methods of treating haematological disorder(s).

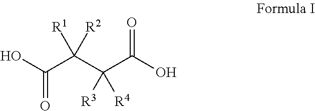

### FIELD OF THE INVENTION

The present invention relates to succinic acid and its derivative(s) of Formula (I), their pharmaceutically acceptable salts, polymorphs, solvates, hydrates, co-crystals, isomers and solvates thereof as an active pharmaceutical ingredient for the treatment of haematological disorder(s). The present invention further relates to compounds of Formula (I), for the treatment of disorders selected from dengue, idiopathic thrombocytopeniarpura (ITP), anaemia and chemotherapy induced myelosuppression. The present invention also relates to use of succinic acid for the treatment of haematological disorder(s), wherein succinic acid is extracted from Papaya leaves. The present invention also provides pharmaceutical compositions comprising succinic acid and its derivative(s) of Formula I and methods of treating haematological disorder(s).

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Succinic acid is a four-carbon dicarboxylic acid, it takes the form of an anion, succinate, which has multiple biological roles as a metabolic intermediate. The anion, succinate, an intermediate of the citric acid cycle and succinic acid have a wide industrial application. Succinic acid is commonly used as a flavoring agent for food and beverages. It is widely used as an intermediate for dyes, perfumes, lacquers, photographic chemicals, alkyd resins, plasticizers, metal treatment chemicals, and coatings. It is used as a precursor for many commercially important chemicals, including 1,4-butanediol, lactic acid and 1,3-propanediol and as a monomer for polymer synthesis.

Succininc acid is also used in the manufacture of medicines such as sedatives, antispasmers, antiplegm, antiphogistic, anthoers, and contraceptives. Succinic acid or succinate are reported to be useful in symptoms related to menopause such as hot flashes and irritability. Succinate is also applied to the skin for arthritis and joint pain. Recently succinate is also reported to have cardio protective effect against ischemia/reperfusion injury (Surgery Today. vol. 28, no. 5, pages 522-528 (1998)). Some other applications include as disclosed in WO200059499, methods of inhibiting protein tyrosine phosphatase activity and of treating disease states caused by dysfunctional signal transduction, which comprises administering succinic acid or a pharmaceutically acceptable salts thereof alone or in combination with activators of protein tyrosine kinases, preferably with insulin. Succinic acid or succinate are involved in several chemical processes in the body and hence commonly used in many supplements.

However, succinic acid or its derivative(s) appear to be underutilized as a pharmaceutical ingredient. Besides, succinic acid is widely available and is produced on a large scale for human use synthetically or converted from biomass via fermentation. Further, succinic acid is usually regarded to be a very safe and effective and typically is very well tolerated.

Thus, succinic acid or derivative(s) thereof, which are so commonly available and well tolerated still, seem to be underexploited as an active pharmaceutical ingredient. Hence, there is a need to further explore succinic acid and derivative(s) thereof as an active pharmaceutical ingredient in compositions, formulations and in the manufacture of medicaments or nutritional supplement for the treatment of various diseases.

### OBJECTS OF THE INVENTION

An object of the present invention is to provide succinic acid or derivative(s) thereof, as an active pharmaceutical ingredient for treatment of haematological disorder(s).

Yet another object of the present invention is to provide succinic acid or derivative(s) thereof, for the manufacture of medicaments or nutritional supplement for the treatment of haematological disorder(s).

Another object of the present invention is to provide succinic acid or derivative(s) thereof, for the manufacture of medicaments for the treatment of disorder selected from dengue, idiopathic thrombocytopeniarpura (ITP), anaemia and chemotherapy induced myelosuppression.

Another object of the present invention is to provide succinic acid extracted from Papaya leaves and its method of extraction.

### SUMMARY OF THE INVENTION

This summary is provided to introduce a selection of concepts in a simplified form that are further described below in Detailed Description section. This summary is not intended to identify key features or essential features of the claimed subject matter, nor is it intended to be used as an aid in determining the scope of the claimed subject matter.

The present invention relates to use of Succinic acid and its derivatives for the treatment of haematological disorders. Specifically, the present invention relates to Succinic acid and its derivatives of Formula (I), their pharmaceutically acceptable salts, polymorphs, solvates, hydrates, co-crystals, isomers and solvates thereof,

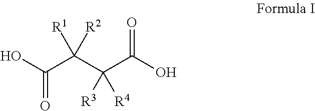

### Wherein:

R¹, R², R³ and R⁴ are independently selected from the group consisting of H, C₁₋₁₂ alkyl —OH, —NH₂, —F, —Cl, 1'Br and —I;
R¹ and R² or R³ and R⁴ can independently represent oxo group (—C═O), for the treatment of haematological disorders.

Another aspect of the present invention relates to use of compounds of Formula I, their pharmaceutically acceptable salts, polymorphs, solvates, hydrates, co-crystals, isomers and solvates thereof, for the treatment of haematological disorders selected from dengue, idiopathic thrombocytopenia purpura (ITP), anaemia chemotherapy induced myelosuppression and the like.

In another aspect, the present invention relates to succinic acid and its derivative(s) of Formula (I), their pharmaceutically acceptable salts, polymorphs, solvates, hydrates, co-crystals, isomers and solvates thereof, as an active pharmaceutical ingredient for the treatment of haematological disorders selected from dengue, idiopathic thrombocytopenic purpura (ITP), anaemia chemotherapy induced myelosuppression and the like.

A further aspect of the present invention is the use of a compound of Formula I for the manufacture of a medicament for the treatment of haematological disorders selected from dengue, idiopathic thrombocytopenia purpura (ITP), anaemia chemotherapy induced myelosuppression and the like in a subject in need thereof.

Another aspect of the present invention provides pharmaceutical compositions, containing compounds of Formula I, their pharmaceutically acceptable salts, polymorphs, solvates, hydrates, co-crystals, isomers and solvates thereof, in combination with one or more pharmaceutically acceptable carrier(s), adjuvants and vehicles.

Yet another aspect of the invention is to provide methods of using the compounds of Formula I of the present invention or compositions comprising the compounds of Formula I for the treatment of haematological disorders selected from dengue, idiopathic thrombocytopenia purpura (ITP), anaemia chemotherapy induced myelosuppression and the like, which comprises administering to a subject in need thereof the compounds of Formula I or compositions comprising a pharmaceutically effective amount of the compounds of Formula I.

Another aspect of the present invention relates to use of compounds of Formula I, their pharmaceutically acceptable salts, polymorphs, solvates, hydrates, co-crystals, isomers and solvates thereof, for inducing the differentiation of thrombocytes and megakaryocytes from the hematopoietic stem cell(HSCs).

Yet another embodiment of the present invention is to provide method for the treatment of haematological disorders selected from dengue, idiopathic thrombocytopenia purpura (ITP), anaemia chemotherapy induced myelosuppression and the like, comprising administering a pharmaceutically effective amount of the compounds of Formula I to a subject in need thereof.

Other aspects of the invention will be set forth in the description which follows, and in part will be apparent from the description, or may be learnt by the practice of the invention.

### BRIEF DESCRIPTION OF DRAWINGS THE INVENTION

The following drawings form part of the present specification and are included to further illustrate aspects of the present disclosure. The disclosure may be better understood by reference to the drawings in combination with the detailed description of the specific embodiments presented herein.

FIG. 1: is a flow diagram of process for preparing papaya leaf extract.

FIG. 2: is a bar graph showing bioassay with fractions from Prep-HPLC run.

FIG. 3: is a bar graph showing results of bioassay using active compound.

FIG. 4: is a structural elucidation using ¹H-NMR

FIG. 5: is a structural elucidation using ¹³C-NMR.

FIG. 6: is a graphical depiction of mass analysis of the active fractions.

FIG. 7: is a Spiked ¹H NMR of purified compound with commercial succinic acid.

FIG. 8: is a bar graph depicting bioassay with sodium succinate.

FIG. 9: is a bar graph depicting bioassay with different salts of succinate

FIG. 10: is a graph depicting effect of succinate to release the cells from G0:
A. Asynchronously growing human diploid fibroblasts
B. Asynchronously growing human diploid fibroblasts induced to enter quiescence by incubating them in a mitogen-deprived medium for 48 hrs.
C. Asynchronously growing human diploid fibroblasts induced to enter quiescence by incubating them in a mitogen-deprived medium in the presence of succinate for 48 hrs.

FIG. 11: is a graph depicting comparison of animal study in a group treated with succinic acid and control group.

### DETAILED DESCRIPTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a.” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Various terms as used herein are shown below. To the extent a term used in a claim is not defined below, it should be given the broadest definition persons in the pertinent art have given that term as reflected in printed publications and issued patents at the time of filing.

The term succinic acid as used herein refers to water-soluble, colourless dicarboxylic acid. The term succinate as used herein refers to an anion of succinic acid, which is an intermediate of the citric acid or TCA cycle and is capable of donating electrons to the electron transfer chain.

Dengue as referred herein refers to a mosquito-borne viral disease caused by dengue viruses of the family Flaviviridae. Mostly occurring in tropical and subtropical areas, the dengue virus is spread by several species of mosquito, especially by Aedes aegypti. The symptoms include headache, joint pains, high fever, vomiting skin rashes etc. In some cases, the patient may develop very low platelet counts that may lead to life-threatening condition known as haemorrhagic dengue fever.

Idiopathic thrombocytopenia purpura (ITP) as used in refers to a bleeding disorder resulted due to the destruction of platelets by immune system. This in turn led to easy or excessive bruising and bleeding.

Anaemia as used in refers to a condition in which there is a deficiency of red cells or haemoglobin (Hb) in the blood. As a result insufficient oxygen is carried around in the bloodstream. The common symptoms include lethargy, palpitations, headache, breathlessness etc.

Myelosuppression as used in refers to bone marrow suppression. A condition where bone marrow activity is decreased resulting in reduced production red blood cells, white blood cells, and platelets. In majority of cases, Myelosuppression appears as a side effect of chemotherapy during cancer treatments.

The term “alkyl” as used herein alone or as part of another group refers to a straight or branched chain aliphatic hydrocarbon chain, having from 1 to 12 carbon atoms. Examples of alkyl include, but are not limited to methyl, ethyl, n-propyl, isopropyl, n-butyl, n-pentyl, t-butyl and the like. Alkyl groups may further be substituted with one or more suitable substituents.

The term “oxo” refers to carbonyl group represented as >C═O.

“Subject” includes humans or non-human mammals (e.g., dogs, cats, rabbits, cattle, horses, sheep and the like).

The term “therapeutically effective amount” means the amount of a compound that, when administered to a subject for treating a disease, is sufficient to effect such treatment for the disease. The “therapeutically effective amount” will vary depending on the compound, the disease and its severity, weight, physical condition and responsiveness of the subject to be treated, among other factors.

A “pharmaceutically acceptable salt” refers to salts prepared from pharmaceutically acceptable non-toxic bases or acids including inorganic or organic bases and inorganic or organic acids. A “pharmaceutically acceptable salt” also encompasses any compound according to the present invention that is utilized in the form of a salt thereof, especially where the salt confers on a compound improved pharmacokinetic properties as compared to the free form of compound or a different salt form of the compound.

The “pharmaceutically acceptable solvates” refer to solvates with water (i.e., hydrates) or pharmaceutically acceptable solvents, for example, ethanol and the like.

Asymmetric centres may exist in the compounds of the present invention. The compounds of Formula I may have one or more stereogenic centres and so can exhibit optical isomerism. All such isomers including enantiomers, diastereomers, and epimers are included within the scope of this invention. Furthermore, the invention includes such compounds as single isomers (R and/or S) and as mixtures, including racemates. If desired, racemic mixtures of the compounds may be separated so that the individual enantiomers are isolated. The separation may be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. Starting materials of particular stereochemistry may either be commercially available or may be made by the methods described herein and resolved by techniques well known in the art. The independent syntheses of these diastereomers or their chromatographic separations may be achieved as known in the art by appropriate modifications.

Certain compounds according to Formula I, can also exist as tautomers, which have different points of attachment of hydrogen accompanied by one or more double bond shifts. These tautomers, either separately or as mixtures, are also considered to be within the scope of the invention.

The present invention relates to use of Succinic acid and its derivatives for the treatment of haematological disorders. Specifically, the present invention relates to Succinic acid and its derivatives of Formula (I), their pharmaceutically acceptable salts, polymorphs, solvates, hydrates, co-crystals, isomers and solvates thereof,

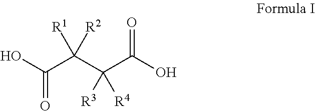

### Wherein:

R¹, R², R³ and R⁴ are independently selected from the group consisting of H, C₁₋₁₂ alkyl —OH, —NH₂, —F, —Cl, —Br and —I;
R¹ and R² or R³ and R⁴ can independently represent oxo group (—C═O), for the treatment of haematological disorders.

Another embodiment of the present invention relates to use of compounds of Formula I, their pharmaceutically acceptable salts, polymorphs, solvates, hydrates, co-crystals, isomers and solvates thereof, for the treatment of haematological disorders selected from dengue, idiopathic thrombocytopenia purpura anaemia (ITP), chemotherapy induced myelosuppression and the like.

In one embodiment, the present invention relates to compounds of Formula I, their pharmaceutically acceptable salts, polymorphs, solvates, hydrates, co-crystals, isomers and solvates thereof, selected from but not limited to Succinic acid, sodium succinatae, 2-chloro-3-fluoro Butanedioic acid, 2,2-difluro Butanedioic acid, 2-amino-3-chloro Butanedioic acid, 2-chloro-3-oxo Butanedioic acid, 2-fluoro-3-oxo Butanedioic acid, 2,3-dichloro-2,3-di fluoro Butanedioic acid, 2,2,3-trifluoro-Butanedioic acid, 2-chloro-3-fluoro Butanedioic acid; and 2,3-dihydroxy Butanedioic acid, and their use for the treatment of haematological disorders selected from dengue, idiopathic thrombocytopenia purpura (ITP), anaemia chemotherapy induced myelosuppression and the like.

In another embodiment, the compounds of the present invention may be synthetically synthesized or obtained from a natural source.

In another embodiment, the compounds of the present invention may commercially available or prepared by techniques known in the art and familiar to the skilled organic chemist or extracted from a plant source or obtained by fermentation using biomass.

Carica papaya (family Caricarceae, papaya), a species native from Mexico and northern South America, has become naturalized throughout the tropical and subtropical regions of the world. Though, mostly being cultivated for the consumption as fresh fruit, the use of the different parts of the plant such as fruit, seed, leaves and root have also been mentioned in ancient texts and in alternative medicines. Recently, several studies from Asian sub-continent have reported the use of papaya leaves for the diseases like dengue, idiopathic thrombocytopenia purpura etc. In majority of these studies, the application of crude papaya leaves extract could efficiently increase the thrombocyte count in the blood in rodent and murine models. However, the identification of the entire spectrum of biologically active compound/s from those leaves extract is still elusive. The present invention discloses the identification of active compounds from the crude leaves, useful for treating haematological disorders, wherein the extract was analyzed utilizing various bio-analytical tools.

In another preferred embodiment, the present invention relates to succinic acid, useful for the treatment of haematological disorders selected from dengue, idiopathic thrombocytopenia purpura (ITP), anaemia chemotherapy induced myelosuppression and the like, wherein the succinic acid can be extracted from aqueous extract of Papaya leaves.

In yet another embodiment, the present invention relates to the compounds of Formula I, useful for the treatment of haematological disorders selected from dengue, idiopathic thrombocytopenia purpura (ITP), anaemia chemotherapy induced myelosuppression and the like, wherein the compounds may be obtained by combination of synthetic sources and from extraction or isolation in pure form from plant source, for example from papaya leaves.

In another embodiment, the present invention relates to use of succinic acid and its derivative(s) of Formula (I), their pharmaceutically acceptable salts, polymorphs, solvates, hydrates, co-crystals, isomers and solvates thereof, as an active pharmaceutical ingredient, for the treatment of haematological disorders selected from dengue, idiopathic thrombocytopenia purpura (ITP), anaemia chemotherapy induced myelosuppression and the like.

Another embodiment of the present invention relates to use of compounds of Formula I, their pharmaceutically acceptable salts, polymorphs, solvates, hydrates, co-crystals, isomers and solvates thereof, for inducing the differentiation of thrombocytes and megakaryocytes from the hematopoietic stem cell (HSCs).

A further embodiment of the present invention relates to the use of a compound of Formula I, for the manufacture of a medicament for the treatment of haematological disorders selected from dengue, idiopathic thrombocytopenia purpura (ITP), anaemia chemotherapy induced myelosuppression and the like in a subject in need thereof.

Another embodiment of the present invention relates to the use of a compound of Formula I, selected from but not limited to Succinic acid, sodium succinatae, 2-chloro-3-fluoro Butanedioic acid, 2,2-difluro Butanedioic acid, 2-amino-3-chloro Butanedioic acid, 2-chloro-3-oxo Butanedioic acid, 2-fluoro-3-oxo Butanedioic acid, 2,3-dichloro-2,3-di fluoro Butanedioic acid, 2,2,3-trifluoro-Butanedioic acid, 2-chloro-3-fluoro Butanedioic acid; and 2,3-dihydroxy Butanedioic acid, for the manufacture of a medicament for the treatment of haematological disorders selected from dengue, idiopathic thrombocytopenia purpura (ITP), anaemia chemotherapy induced myelosuppression and the like in a subject in need thereof.

Another embodiment of the present invention provides pharmaceutical compositions containing compounds of Formula I, their pharmaceutically acceptable salts, polymorphs, solvates, hydrates, co-crystals, isomers and solvates thereof, in combination with one or more pharmaceutically acceptable carrier(s), adjuvants and vehicles.

Another embodiment of the present invention relates to the use of a compound of Formula I, for the manufacture of a medicament for the treatment of haematological disorders selected from dengue, idiopathic thrombocytopenia purpura (ITP), anaemia chemotherapy induced myelosuppression and the like in a subject in need thereof, wherein the medicament is administered orally, parenterally or topically.

The present invention provides pharmaceutical compositions, comprising compounds of Formula I or their pharmaceutically acceptable salts, polymorphs, solvates, hydrates, co-crystals. isomers and solvates thereof together with one or more pharmaceutically acceptable carriers comprising excipients and auxiliaries, which facilitate processing of the active compound into preparations, which can be used pharmaceutically. The pharmaceutical compositions may be in the forms normally employed, such as tablets, capsules, powders, syrups, solutions, suspensions, emulsions, pills, granules, suppositories, pellets, depot formulations and the like, may contain flavourants, sweeteners etc in suitable solid or liquid carriers or diluents, or in suitable sterile media to form injectable solutions or suspensions. Such compositions typically contain from 0.1 to 99.9% by weight of active compound, the remainder of the composition being pharmaceutically acceptable carriers, diluents or solvents.

The pharmaceutical compositions of the present invention can be manufactured by the processes well known in the art, for example, by means of conventional mixing, dissolving, dry granulation, wet granulation, dragee-making, levigating, emulsifying, encapsulating, entrapping, lyophilizing processes or spray drying. The compounds or the pharmaceutical compositions comprising such compounds of the present invention may be administered in the form of any pharmaceutical formulation. The pharmaceutical formulation will depend upon the nature of the active compound and its route of administration. Any route of administration may be used, for example oral, buccal, pulmonary, topical, parenteral (including subcutaneous, intramuscular and intravenous), transdermal, ocular (ophthalmic), by inhalation, intranasal, transmucosal, implant or rectal administration. Preferably the compounds of the present invention are administered orally, parenterally or topically.

An embodiment of the present invention provides a therapeutically effective amount of a compound of Formula I, or its pharmaceutically acceptable derivatives, tautomeric forms, stereoisomers, polymorphs, prodrugs, metabolites, salts or solvates thereof, for use as a pharmaceutical composition.

In another embodiment, the amount of the compounds having the Formula I according to the present invention to be incorporated into the pharmaceutical compositions of the present invention can vary over a wide range depending on known factors such as, for example, the disorder to be treated, the severity of the disorder, the patient's body weight, the dosage form, the chosen route of administration and the number of administration per day. Typically, the amount of the compound of Formula I in the pharmaceutical compositions of the present invention will range from approximately 0.01 mg to about 500 mg. In an embodiment, the daily dose of composition comprising the compounds having the Formula I is in the range of about 0.01 mg/kg to about 100 mg/kg based on the body weight of the subject in need thereof which may be administered as a single or multiple doses.

Yet another embodiment of the present invention is to provide method for the treatment of haematological disorders selected from dengue, idiopathic thrombocytopenia purpura (ITP), anaemia chemotherapy induced myelosuppression and the like, comprising administering a pharmaceutically effective amount of the compounds of Formula I to a subject in need thereof.

Another embodiment of the present invention is to provide method for the treatment of haematological disorders selected from dengue, idiopathic thrombocytopenia purpura (ITP), anaemia chemotherapy induced myelosuppression and the like, comprising administering a pharmaceutically effective amount of the compounds of Formula I to a subject in need thereof, wherein the compound can be selected from Succinic acid (Butanedioic acid), sodium succinate, 2-chloro-3-fluoro Butanedioic acid, 2,2-difluro Butanedioic acid, 2-amino-3-chloro Butanedioic acid, 2-chloro-3-oxo Butanedioic acid, 2-fluoro-3-oxo Butanedioic acid, 2,3-dichloro-2,3-di fluoro Butanedioic acid, 2,2,3-trifluoro-Butanedioic acid, 2-chloro-3-fluoro Butanedioic acid and 2,3-dihydroxy Butanedioic acid.

Yet another embodiment of the present invention is to provide method for the treatment of haematological disorders selected from dengue, idiopathic thrombocytopenia purpura (ITP), anaemia chemotherapy induced myelosuppression and the like, comprising administering a pharmaceutically effective amount of the compounds of Formula I to a subject in need thereof, wherein compounds of Formula (I) can be obtained from synthetic source, a natural source, by fermentation using biomass, in the form of extract or isolated in pure form from plant source or by combination of these processes.

Still another embodiment of the present invention is to provide method for the treatment of haematological disorders selected from dengue, idiopathic thrombocytopeniaurpura (ITP), anaemia chemotherapy induced myelosuppression and the like, comprising administering a pharmaceutically effective amount of the compounds of Formula I to a subject in need thereof, wherein succinic acid is obtained from aqueous extract of Papaya leaves.

Another embodiment of the present invention is to provide method for the treatment of haematological disorders selected from dengue, idiopathic thrombocytopenia purpura (ITP), anaemia chemotherapy induced myelosuppression and the like, comprising administering a pharmaceutically effective amount of the compounds of Formula I to a subject in need thereof, for inducing the differentiation of thrombocytes and megakaryocytes from the hematopoietic stem cell (HSCs).

In yet another aspect, the present disclosure provides a composition comprising compounds of Formula (I), their pharmaceutically acceptable salts, polymorphs, solvates, hydrates, co-crystals, isomers and solvates thereof, optionally containing an additional therapeutic agent selected from paclitaxel, docetaxel, colchicines, vincristine, vinblastine, doxorubicin, daunorubicin, dactinomycin, 5-Fluorouracil (5-FU), methotrexate, 6-thiopurines, mercaptopurine, thioguanine, cladribine, pentostatin, cytarabine, azactidine, fludarabine, gemcitabine or hydroxyurea, for the treatment of disorder selected from haematological disorders, dengue, idiopathic thrombocytopenia purpura (ITP), anaemia and chemotherapy induced myelosuppression.

In another embodiment, the present invention relates to use of compound of Formula (I) their pharmaceutically acceptable salts, polymorphs, solvates, hydrates, co-crystals, isomers and solvates thereof, as an active pharmaceutical ingredient, or composition, or formulation, for inducing the differentiation of thrombocytes and megakaryocytes from the hematopoietic stem cell(HSCs).

While the foregoing describes various embodiments of the disclosure, other and further embodiments of the disclosure may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### EXAMPLES

The disclosure is further illustrated by the following examples which in no way should be construed as being further limiting. One skilled in art will readily appreciate that the specific methods are results described are merely illustrative.

### Abbreviations

rhGMCSF: recombinant human Granulocyte-macrophage colony-stimulating factor
IL3: Interleukin 3
FBS: Foetal Bovine Serum
RP-HPLC: Reversed-Phase High-Performance Liquid Chromatography
Prep-HPLC: Preparative High-Performance Liquid Chromatography
LC-MS: Liquid Chromatography-Mass Spectrometry
NMR: Nuclear Magnetic Resonance
P.O.: Per os
HSCs: Hematopoietic Stem Cells
CBC: Complete Blood Counts

### Example 1: Preparation of Freeze Dried Extract From Papaya Leaves

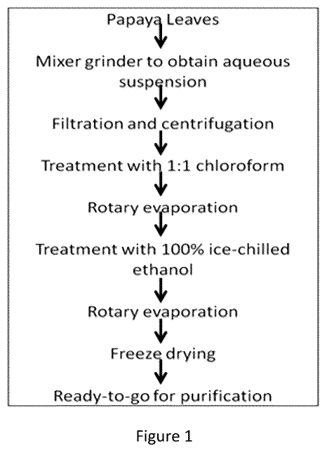

Freshly harvested papaya leaves obtained from Panchgani, Maharashtra, India were chopped into pieces after removing the main stems. The leaves were thoroughly washed in water followed by grinding into a mixer grinder to make an aqueous suspension. The suspension was filtered through muslin cloth followed by high-speed centrifugation (15,000×g for 30 minutes) to remove any debris. The aqueous extract was subjected to chloroform treatment (1:1 v/v. 3×) to separate chlorophyll and other organic components. Trace amount of chloroform was removed from the aqueous part by rotary evaporation. The aqueous part was then subjected to ethanol precipitation by treating it with 100% ice-chilled ethanol to precipitate proteins and nucleic acids. The trace amount of ethanol was further removed by rotary evaporation followed by freeze drying the aqueous part in a freeze drier. The dried material was weighed and stored at 4° C. (FIG. 1). 5 kg of dried papaya leaves yield 100 gm of freeze dried extract. The freeze dried extract was used to check the biological activity in cell lines and then further processed to purify the active ingredients using different HPLC techniques which yielded 5 mg of purified compound.

### Example 2: In-Vitro Bioassay in TF1 Cell Lines Using Freeze Dried Extract With Different HPLC Fractions

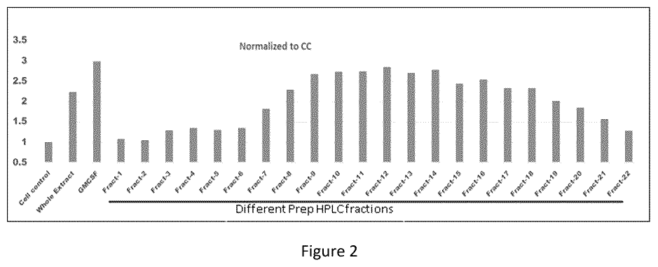

The TF-1 cell line (ATCC-CRL 2003) which was derived from a patient with erythro-leukaemia, shows complete growth dependency on GMCSF or IL-3. For the present study, TF-1 cells were used for measuring the in-vitro activity in the freeze dried extract, different HPLC fractions, purified compounds and succinic acid derivatives. The cells were grown and maintained in RPMI-1640 complete medium supplemented with 2 ng/ml rhGMCSF. For proliferation assay, TF-1 cells were plated at 10,000 cells/well in a 96-well plate. Cells were cultured overnight in RPMI 1640 containing 5% FBS after removing rhGMCSF from the cells by repeated washing. Cells were then treated with freeze dried extract, different HPLC fractions, purified compound and Succinic acid derivatives for 48 h. The cells treated with rhGMCSF (2 ng/ml) were used as positive control, whereas cells without any growth factors were used as negative control. The cellular proliferation was measured using the alamar Blue cell viability reagent. The 96-well plate was read at an excitation wavelength of 530 nm and emission wavelength of 590 nm. The relative fluorescence units obtained are directly proportional to the number of live cells. The freeze dried extract from Example 1 was fractionated using Prep-HPLC. The different fractions collected along with freeze dried extract were directly used in TF-1 cells to examine the proliferation potential. rhGM-CSF used as positive control, whereas cells without any growth factors were used as negative control. Values obtained from different fractions were normalized to cell control value. The cellular proliferation stimulated by different fractions showed a Poisson distribution pattern. The result indicates the presence of bioactive compound in fractions 7 to fraction 21. (FIG. 2)

### Example 3: Purification of the Active Compound From the Freeze Dried Extract and Bioassay Using the Active Compound

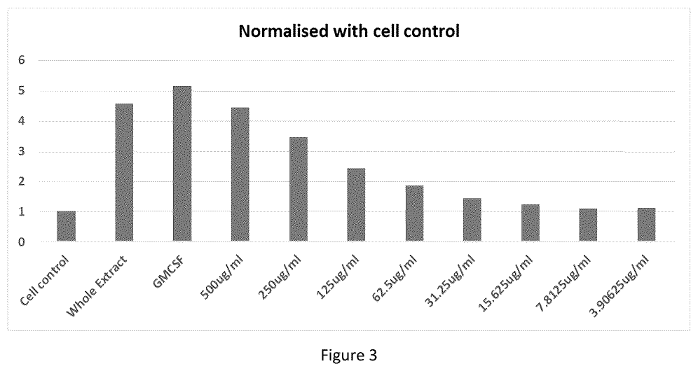

Freeze dried extract was reconstituted in mobile phase A (0.1% acetic acid in water) and loaded onto a 50×250 mm prep-HPLC Cis column. Post loading the column was washed for 3 column volumes with mobile phase A followed by elution with mobile phase B (100% acetonitrile containing 0.1% acetic acid). The active fractions of example 2 were pooled, dried and subjected to further purification via analytical HPLC and UPLC techniques to obtain active compound. The final active compound was dried and tested for activity in TF-1 cells using different dilutions. A dose dependent proliferation was observed with the pure compound (FIG. 3).

### Example 4: Characterization of the Active Fractions

For structural elucidation, the active compound was analysed by melting point, ¹H-NMR, ¹³C-NMR and LC-MS.

Melting point of pure compound form papaya extract is 184-186° C. which was same as the melting point of commercial succinic acid.

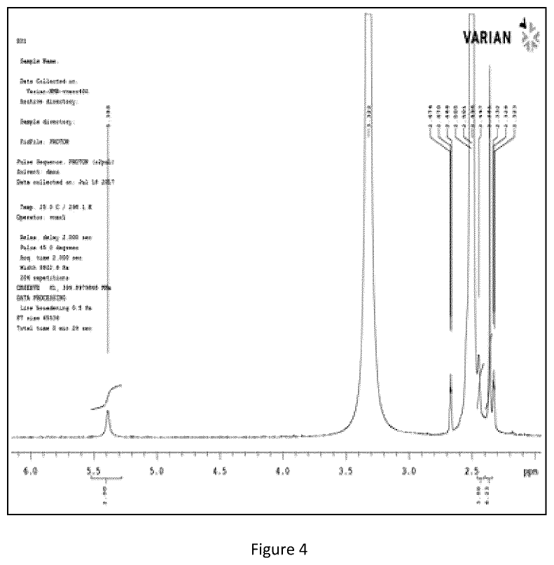

The active compound was analyzed by ¹H NMR. Based on the analysis the pure compound showed the structural similarity with succinic acid (FIG. 4).

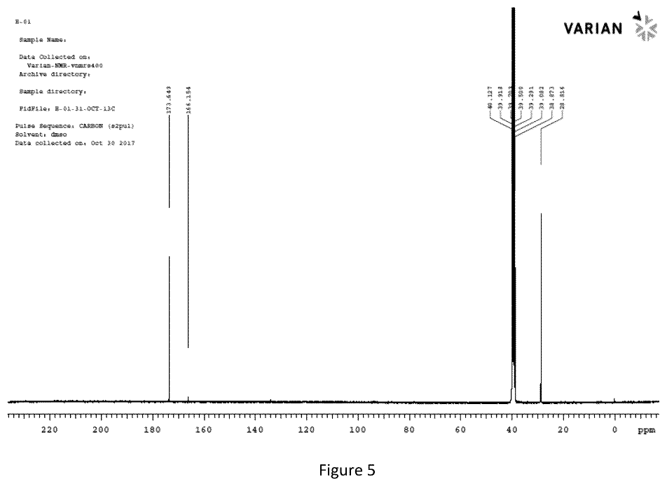

The pure compound was analyzed by ¹³C NMR. Based on the analysis the pure compound showed the structural similarity with succinic acid (FIG. 5).

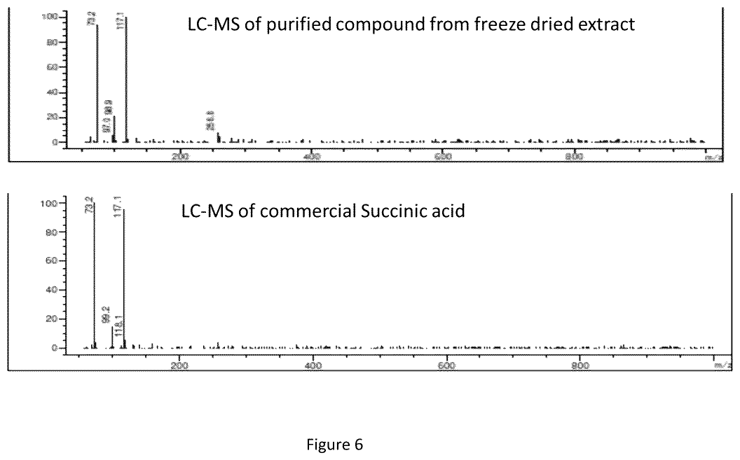

The pure fraction was subjected to LC-MS analysis in negative mode. MS analysis revealed the major peak of 118 Dalton (m/z: 117 in negative mode). Similarly, the commercial succinic acid also showed the similar LC-MS profile as the pure compound (FIG. 6).

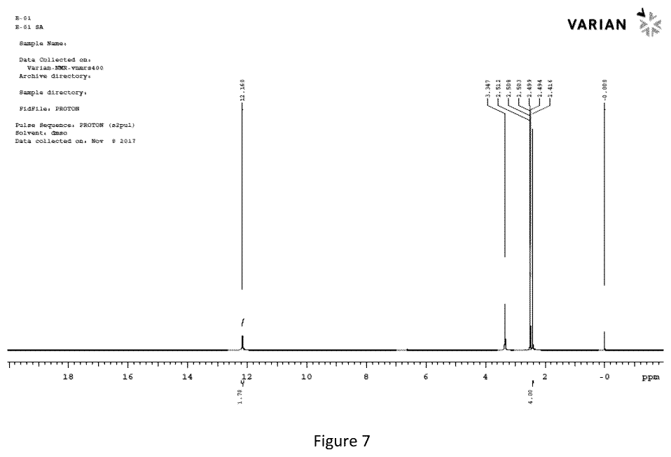

The ¹H NMR of active compound from papaya extract was spiked with the commercial Succinic acid. The peaks are overlapping to each other which confirms the active compound extracted from papaya leaves is Succinic Acid (FIG. 7).

Therefore, the active compound isolated from papaya leaves is confirmed as succinic acid.

### Example 5: Bioassay With Commercial Sodium Succinate

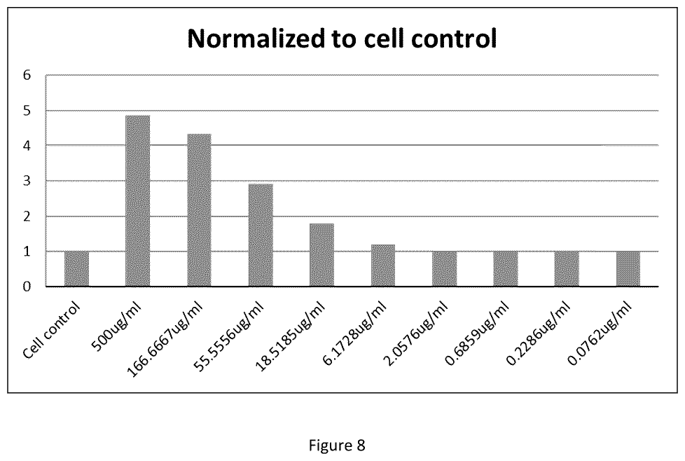

To further validate the NMR and LC-MS data, Sodium Succinate was procured and used to test the proliferation potential of succinate. We could reproduce the similar proliferation activity in a dose dependent manner in the TF-1 cells as it was shown by the active compound (FIG. 3) isolated from papaya leaf extract (FIG. 8).

### Example 6: Bioassay With Different Salts of Succinic Acid

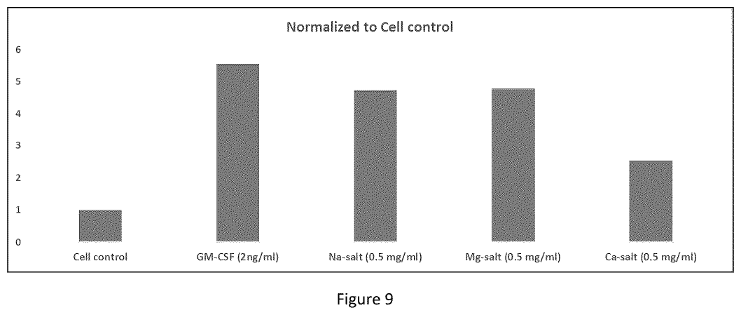

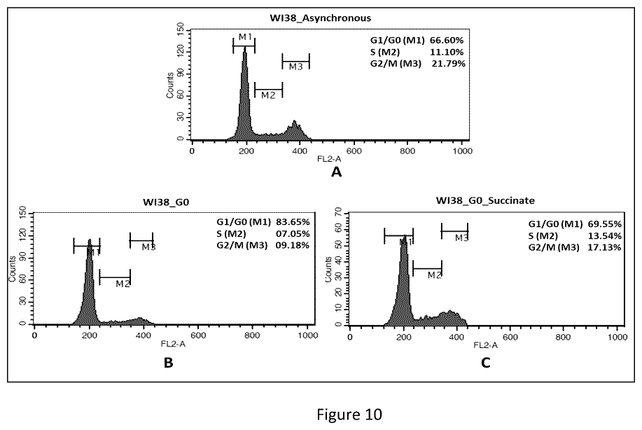

Na, Mg and Ca salts of succinic acids were procured and used to test the proliferation potential of the different salts of succinate. All the salt forms tested showed proliferation activity in TF-1 cells (FIG. 9).

### Example 7: Succinate Induces Cell Proliferation in a Cell Culture Model

Human diploid, primary fibroblast WI38 cells were utilized to induce quiescence by serum deprivation. Briefly, WI38 cells were grown in a media containing 0.1% foetal bovine serum (FBS) for 48 hrs to cause a reversible cell cycle arrest or quiescence or G0. To understand the role of succinate, asynchronously growing human diploid fibroblasts (FIG. 10A) were induced to enter quiescence by incubating them in a mitogen-deprived medium (0.1% foetal bovine serum) in the presence of succinate for 48 h. In the presence of succinate. WI38 cells entered in the cell cycle even in the absence of mitogens in the media (FIG. 10C). The control group growing in the presence of mitogen-deprived medium shows cell cycle arrest or quiescence (FIG. 10B). This indicates a role of succinate to induce cells to enter in cell division from a quiescence stage. Here to mention, the hematopoietic stem cells (HSCs) are quiescent most of the time and depending upon external or internal cues undergo a limited number of self-renewing divisions with maintenance of multipotency.

### Example 8: Animal Study Using Succinic Acid

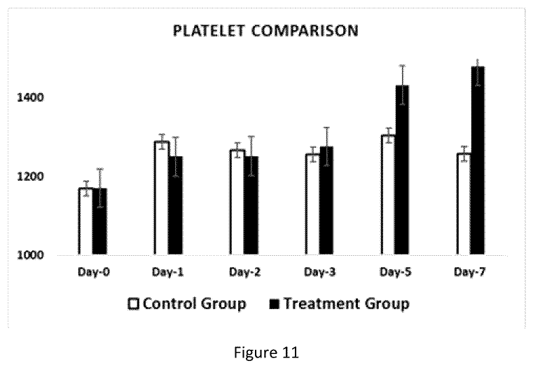

A preliminary study was conducted in rats using pure succinic acid (from commercial source). Six animals were used in each group (control and treated). The treated group received a dose of 100 mg/kg succinic acid twice daily P.O. whereas the control group received water of same volume as in treated twice daily. CBC (Complete Blood counts) was performed from the blood withdrawn from individual rats. Dosing of rat with 100 mg/kg succinic acid twice daily P.O. for 7 days could substantially increase the platelet counts (FIG. 11).

### Example 9: RNA-seq Analysis of Gene Expression Changes in TF-1 Cells

For RNA-seq analysis, TF-1 cells were either treated with the positive control GMCSF (GM) or with succinate (E01). Untreated TF-1 cells (CC) were used as a control to determine basal gene expression. RNA-seq was performed in triplicates from all the three groups. The gene expression from E01 or GM group was normalised to the expression of CC. Several genes related to platelet differentiation pathway in succinate treated cells were identified (e.g., GP6, ANGPT1, ARRB, CD36, PECAM1 etc).

### Example 10: Evaluation of Biological Activity of Derivative(s) of Succinic Acid

2.2-difluro Butanedioic acid was procured from commercial sources and tested for bioassay as described in example 2. This derivative showed similar activity compared to sodium salt of Succinic acid.

### Advantages of the Present Invention

The present invention provides succinic acid and derivatives of Formula I, as an active pharmaceutical ingredient for treatment of various diseases.

The present invention provides succinic acid and derivatives of Formula I, for the manufacture of medicaments for the treatment of haematological disorder(s).

The present invention succinic acid and derivatives of Formula I, for the manufacture of medicaments for the treatment of disorder selected from dengue, idiopathic thrombocytopeniarpura (ITP), anaemia and chemotherapy induced myelosuppression.

The present invention provides use of succinic acid and derivatives of Formula I, extracted from Papaya leaves for the manufacture of medicaments or nutritional supplement for the treatment of various diseases.

1-16. (canceled)

17. A method for the treatment of haematological disorders selected from dengue, idiopathic thrombocytopeniaurpura (ITP), anaemia, and chemotherapy induced myelosuppression, comprising administering a pharmaceutically effective amount of the compounds of Formula I, their pharmaceutically acceptable salts, polymorphs, solvates, hydrates, co-crystals, isomers and solvates to a subject in need thereof, 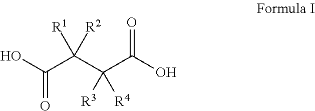
Wherein:
R¹, R², R³ and R⁴ are independently selected from the group consisting of H, C₁₋₁₂ alkyl, —OH, —NH₂, —F, —Cl, —Br and —I; or
R¹ and R² or R³ and R⁴ can independently represent oxo group (—C═O).

18. The method as claimed in claim 1, wherein the compound can be selected from Succinic acid (Butanedioic acid), sodium succinate, magnesium succinate, calcium succinate,2-chloro-3-fluoro Butanedioic acid, 2,2-difluro Butanedioic acid, 2-amino-3-chloro Butanedioic acid, 2-chloro-3-oxo Butanedioic acid, 2-fluoro-3-oxo Butanedioic acid, 2,3-dichloro-2,3-di fluoro Butanedioic acid, 2,2,3-trifluoro-Butanedioic acid, 2-chloro-3-fluoro Butanedioic acid and 2,3-dihydroxy Butanedioic acid.

19. The method as claimed in claim 1, wherein compounds of formula (I) can be obtained from a synthetic source, a natural source, by fermentation using biomass, in the form of an extract or isolated in pure form from plant source or by combination of these processes.

20. The method as claimed in claim 1, wherein succinic acid or its derivatives are administered at a dose of about 5 mg/kg to 200 mg/kg.

21. The method as claimed in claim 4, wherein succinic acid or its derivatives are administered at a dose of about 50 mg/kg to 200 mg/kg.

22. The method as claimed in claim 4, wherein succinic acid or its derivatives are administered in pharmaceutically acceptable liquid or solid carriers or diluents, or in suitable sterile media to form injectable solutions or suspensions.

23. The method as claimed in claim 4, wherein succinic acid or its derivatives contain 0.1 to 99.9% (w/v) as an active compound used in a suitable form of administration.

24. The method as claimed in claim 1, wherein succinic acid or its derivatives are administered at a dose for inducing the differentiation of thrombocytes and megakaryocytes from the hematopoietic stem cells (HSCs).
